(19) United States
(12) Patent Application Publication
(10) Pub. No.: US 2026/0137860 A1
(43) Pub. Date: May 21, 2026

(54) DEVICES, SYSTEMS, AND METHODS FOR VALIDATING AUTOMATED PROGRAMMING REQUESTS

(71) Applicant: CareFusion 303, Inc., San Diego, CA (US)

(72) Inventor: Michael K. WORKMAN, Carlsbad, CA (US)

(21) Appl. No.: 19/120,453

(22) PCT Filed: Oct. 18, 2023
(86) PCT No.: PCT/US2023/035451

§ 371 Date: Apr. 11, 2025

Related U.S. Application Data
(60) Provisional application No. 63/417,662, filed on Oct. 19, 2022.

(51) Int. Cl. A61M 5/142; A61M 5/172; G16H 20/17; G16H 40/67

(52) U.S. Cl. CPC A61M 5/142; A61M 5/172; G16H 20/17; G16H 40/67; A61M 2005/14208; A61M 2205/18; A61M 2205/3327; A61M 2205/3379; A61M 2205/502; A61M 2205/581

(57) ABSTRACT

An infusion device includes a fluid pump and a processor. The processor is configured to receive an automated programming request (APR) from a device manager, where the APR includes an indicated order type. The processor is also configured to determine an implied order type for the APR based in part on whether the fluid pump is active. Additionally, the processor is configured to determine whether the indicated order type is compatible with the implied order type. If the indicated order type is compatible with the implied order type, the processor sends an acknowledgment message to the device manager and programs the fluid pump according to the operating parameters. However, if the indicated order type is incompatible with the implied order type, the processor sends a non-acknowledgment message and an error message to the device manager and rejects the command to automatically program the fluid pump according to the operating parameters.

### TECHNICAL FIELD

The present disclosure relates, generally, to the automated programming of infusion devices and, more specifically, to the validation of automated programming requests for infusion devices.

### BACKGROUND

In the realm of modern healthcare, the precise and controlled administration of medications and fluids is paramount to patient well-being and recovery. Infusion pumps have played a pivotal role in this context, providing a dependable method for the delivery of intravenous fluids. The integration of automated programming has further advanced infusion technologies by enhancing treatment precision and streamlining healthcare procedures.

Notwithstanding the many benefits of automated programming, increased reliance thereon necessitates rigorous validation processes to ensure patient safety and regulatory compliance. Current validation methods are often manual, time-consuming, and prone to human error. There is a need for computer-driven validation systems to enhance the accuracy and reliability of automated programming.

### SUMMARY

The present disclosure provides devices, systems, and methods that address some of the aforenoted problems, as well as other problems associated with the automated programming of infusion devices. These devices, systems, and methods rely on the computer-driven validation of APRs for infusion devices, including various “fail-fast” methodologies, discussed in more detail below. Example implementations of the advancements discussed herein include:

An infusion device includes a fluid pump and a processor. The processor is configured to receive an APR from a device manager. The APR includes (i) an indicated order type, (ii) an identifier of the fluid pump, (iii) operating parameters including a requested fluid type and requested dosage amount, and (iv) a command to automatically program the infusion device according to the operating parameters. The processor is also configured to detect an activity state of the fluid pump, indicating whether the fluid pump is active. Additionally, the processor is configured to determine an implied order type for the APR based in part on the detected activity state. The implied order type includes an initial order, a subsequent order, a modification order, a bolus order, or a secondary order. Further, the processor is configured to determine whether the indicated order type is compatible with the implied order type and the fluid pump based in part on whether the indicated order type matches the implied order type. Moreover, the processor is configured to, responsive to determining that the indicated order type is compatible with the implied order type and the fluid pump, (i) send an acknowledgment message to the device manager and (ii) program the fluid pump according to the operating parameters. Furthermore, the processor is configured to, responsive to determining that the indicated order type is incompatible with the implied order type or the fluid pump, (i) send an error message to the device manager, and (ii) reject the command to automatically program the fluid pump according to the operating parameters.

A computer-implemented method for validating APRs includes receiving an APR from a device manager. The APR includes (i) an indicated order type, (ii) an identifier of a fluid pump of an infusion device, (iii) operating parameters including a requested fluid type and requested dosage amount, and (iv) a command to automatically program the infusion device according to the operating parameters. The method also includes detecting an activity state of the fluid pump, indicating whether the fluid pump is active. Additionally, the method includes determining an implied order type for the APR based in part on the detected activity state. The implied order type includes an initial order, a subsequent order, a modification order, a bolus order, or a secondary order. Further, the method includes determining whether the indicated order type is compatible with the implied order type and the fluid pump based in part on whether the indicated order type matches the implied order type. Moreover, the method includes, responsive to determining that the indicated order type is compatible with the implied order type and the fluid pump, (i) sending an acknowledgment message to the device manager and (ii) programming the fluid pump according to the operating parameters. Furthermore, the method includes, responsive to determining that the indicated order type is incompatible with the implied order type or the fluid pump, (i) sending an error message to the device manager, and (ii) rejecting the command to automatically program the fluid pump according to the operating parameters.

A non-transitory, computer-readable storage medium includes instructions that, when executed by an electronic device, cause the electronic device to receive an APR from a device manager. The APR includes (i) an indicated order type, (ii) an identifier of a fluid pump of an infusion device, (iii) operating parameters including a requested fluid type and requested dosage amount, and (iv) a command to automatically program the infusion device according to the operating parameters. The instructions also cause the electronic device to detect an activity state of the fluid pump, indicating whether the fluid pump is active. Additionally, the instructions cause the electronic device to determine an implied order type for the APR based in part on the detected activity state. The implied order type includes an initial order, a subsequent order, a modification order, a bolus order, or a secondary order. Further, the instructions cause the electronic device to determine whether the indicated order type is compatible with the implied order type and the fluid pump based in part on whether the indicated order type matches the implied order type. Moreover, the instructions cause the electronic device to, responsive to determining that the indicated order type is compatible with the implied order type and the fluid pump, (i) send an acknowledgment message to the device manager and (ii) program the fluid pump according to the operating parameters. Furthermore, the instructions cause the electronic device to, responsive to determining that the indicated order type is incompatible with the implied order type or the fluid pump, (i) send an error message to the device manager, and (ii) reject the command to automatically program the fluid pump according to the operating parameters.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description, below, in conjunction with the following drawings. Like reference numerals refer to corresponding parts throughout the figures and description.

FIGS. 1A and 1B depict an example patient care system that includes infusion pumps mounted to a control unit, according to various aspects of the subject technology.

FIG. 2 depicts an example institutional patient care system of a healthcare organization, according to various aspects of the subject technology.

FIG. 3 depicts an example system for automatically programming a medical device, according to various aspects of the subject technology.

FIG. 4 depicts an example sequence diagram for automatically validating an automated programming request (APR), according to various aspects of the subject technology.

FIG. 5A depicts an example process for validating APRs, according to various aspects of the subject technology. Additionally, FIGS. 5B and 5C depict example processes for determining, respectively, an implied order type of an APR and an active infusion type of a fluid pump, according to various aspects of the subject technology.

FIG. 6 is a conceptual diagram illustrating an example electronic system for validating APRs, according to various aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The following figures illustrate some of the aforenoted devices, systems, and methods, which can be used to supplement or replace manual validation processes for automated programming requests (APRs). These devices, systems, and methods rely on computer-driven validation, which is likely to be more accurate and more efficient than human-driven validation. The APR validation described herein incorporates various fail-fast methodologies, further enhancing the efficiency thereof. Accordingly, the teachings of the present disclosure are likely to improve healthcare environments that rely on the automated programming of infusion devices.

Many of the fail-fast validation methodologies discussed herein involve a comparison between the indicated order type and the implied order type of an APR. As used herein, “indicated order type” refers to an order type explicitly indicated in an APR. For example, when requesting an APR, a user may indicate that a particular type of order is requested. The APR may indicate, for example, whether it is intended to correspond to an initial order (e.g., a new order for an inactive fluid pump), a subsequent order (e.g., an order subsequent to an ongoing infusion), a modification order (e.g., a modification of an ongoing infusion), a bolus order (e.g., a temporary increase in the flow rate of an ongoing infusion), or a secondary order (e.g., an order concurrent with an ongoing infusion).

By contrast, “implied order type” refers to an order type suggested by the operational parameters of the APR, as well as other contextual clues (e.g., whether the target fluid pump is active and what type of infusion therapy it is administering) determined by the infusion device that receives the APR. For example, if the APR designates a channel (e.g., a fluid pump) of the infusion device and the channel is not currently active, then the infusion device may determine that the implied order type is an initial order because there is not currently an active infusion. This determination may be independent of whether the APR explicitly indicates another order type, such as a bolus order or a secondary order.

As noted above, in some implementations, an APR is validated in part by determining whether the indicated and the implied order types of the APR are compatible with each other. For example, if the APR indicates that it is for an initial order and the operating parameters and other contextual clues imply that the APR corresponds to an initial order, then the APR may be validated because the two order types match. However, if the indicated and implied order types are different, then the infusion device may need to determine whether the indicated and implied order types are compatible and/or whether the APR can still be implemented at the infusion device. This compatibility determination may be based, for example, on whether the target fluid pump is active and, if it is, what type of infusion it is administering. Potential infusion types include continuous infusions (e.g., delivery of an amount of a drug over an amount of time), an intermittent infusion (e.g., delivery of an amount of a drug), a fluid infusion (e.g., delivery of a volume of a fluid such as saline), and a drug infusion (e.g., delivery of a volume of a drug such as an anesthetic).

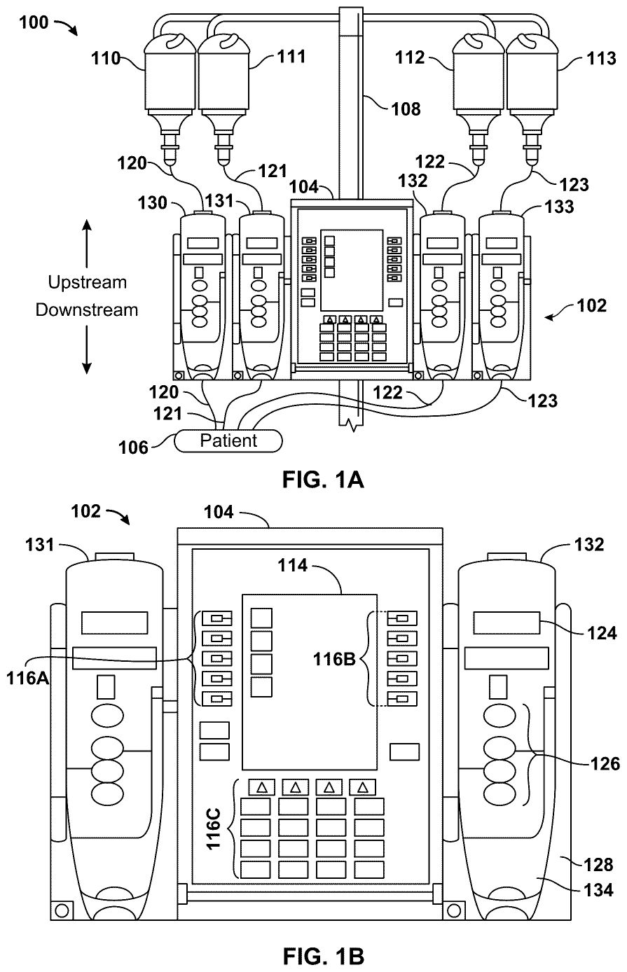

FIGS. 1A and 1B depict an example patient care system 100 that includes infusion pumps 130-133 (131-132 in FIG. 1B) mounted to a control unit 104, according to various aspects of the subject technology. The patient care system 100 shown in FIG. 1A includes four infusion pumps 130-133, each of which is in operative engagement with a respective administration set 120-123 (e.g., silicon tubing). The administration sets 120-123 connect the infusion pumps 130-133 to fluid supplies 110-113, which are inverted and suspended above the infusion pumps 130-133. The fluid supplies 110-113 are depicted as bottles in FIG. 1A, but they may take other forms (e.g., bags). Both the fluid supplies 110-113 and the patient care device 102 are mounted to a roller stand 108.

The fluid supplies 110-113, as well as their orientation (e.g., mount location, mount height, mount type) within the care area, may generate one or more interaction records. The interaction record for a set, for example, may be generated in part by detecting a scannable code associated with the set or by detecting a physical structure on the set that encodes identifying information for the set prior to use.

As shown in FIG. 1A, each administration set 120-123 is connected between a respective fluid supply 110-113 and a patient 106 so that the patient 106 may receive the fluids in any or all the fluid supplies 110-113. Each of the administration sets 120-123 may be identified either actively (e.g., by clinician scanning) or passively (e.g., by wireless or optical detection). Typically, medical administration sets have more parts than are shown in FIG. 1A. Many have check valves, drip chambers, valved ports, connectors, and other devices well known to those skilled in the art. These other devices are not included in the drawings to preserve clarity of illustration.

In the depicted example, a separate infusion pump 130-133 is used to infuse each of the respective fluids of the fluid supplies 110-113 into the patient 106. The infusion pumps 130-133 are flow control devices that will act on the respective tube or fluid conduit of the respective administration set 120-123 to move fluid from the fluid supply 110-113, through the conduit, and to the patient. Because individual infusion pumps 130-133 are used, each of the infusion pumps 130-133 may be individually set to the pumping or operating parameters required for infusing the particular medical fluid from the respective fluid supply 110-113 into the patient at the particular rate prescribed for that fluid by the clinician.

FIG. 1B depicts a portion of the patient care device 102 shown in FIG. 1A. The illustrated patient care device 102 includes the control unit 104, along with two of the infusion pumps 131 and 132 mounted at either side of the control unit 104. In some implementations, the control unit 104 is configured for programming each of the infusion pumps 130-133 (see FIG. 1A). FIG. 1B also shows the displays and controls of the infusion pumps 131 and 132, such as the display 124 and the controls 126 of the infusion pump 132.

Each of the infusion pumps 130-133 may include a door and a handle. For example, infusion pump 132 includes door 128 and handle 134. The handle 134 operates to lock the door 128 in a closed position during operation. The handle 134 also operates unlock and open the door for loading an administration set (e.g., administration set 122) and for accessing the internal pumping and sensing mechanisms of the infusion pump 132. While the door 128 is open, the administration set 122 can be connected with the infusion pump 132. While the door 128 is closed, the administration set 122 is brought into operative engagement with the pumping mechanism, the upstream and downstream pressure sensors, and/or the other equipment of the infusion pump 132.

The infusion pumps 130-133 may also include a display. For example, in the depicted implementation, the display 124 of the infusion pump 132 (e.g., an LED display) is located in plain view on the door 128 and may be used to visually communicate information regarding the infusion pump 132. For example, the display 124 can communicate alert indications (e.g., alarm messages). Additionally, the control keys 126 allow for programming and controlling operations of the infusion pump as desired. In some implementations, the control keys may be presented as interactive elements on the display 124 (e.g., a touchscreen display). The patient care device 102 and/or infusion pump 132 may also include audio alert equipment in the form of a speaker.

The control unit 104 of the patient care device 102 also includes a display 114 for visually communicating various information, such as the operating parameters of a connected pump or alert indications and alert messages. The control unit 104 also includes control keys 116A-C for selecting or setting control parameters and/or options for controlling the control unit 104 and modules connected thereto (e.g., infusion pumps 130-133).

In addition to the display 114 and the control keys 116A-C, the control unit 104 may also include a speaker to provide audible alerts. In some implementations, the display 114 is implemented as a touchscreen display. In such implementations, the control keys 116A-C may be omitted or reduced in number by providing corresponding interactive elements via a graphical user interface presented via the display 114. In some implementations, the control keys 116A-C may select a corresponding option displayed in display 114.

The control unit 104 may also include a communications system by which the control unit 104 can communicate with external equipment, such as a medical facility server, a handheld communication device, a laptop-type computer, or other information device that a clinician may have to transfer information or to download drug libraries to the control unit 104.

The communication module may be used to transfer access or interaction information for clinicians encountering the control unit 104 or a device coupled thereto (e.g., infusion pumps 130-133, or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pumps 130-133, such as in cases where a control unit is not used, or in addition to one with the control unit 104. Further, information input devices need not be hard-wired to medical instruments, information may be transferred through a wireless connection as well. Additionally, other types of modules may be connected to the infusion pumps 130-133 or to the control unit 104 such as a syringe pump module, a patient controlled analgesic module, an end-tidal CO2 monitoring module, an oximeter monitoring module, or the like.

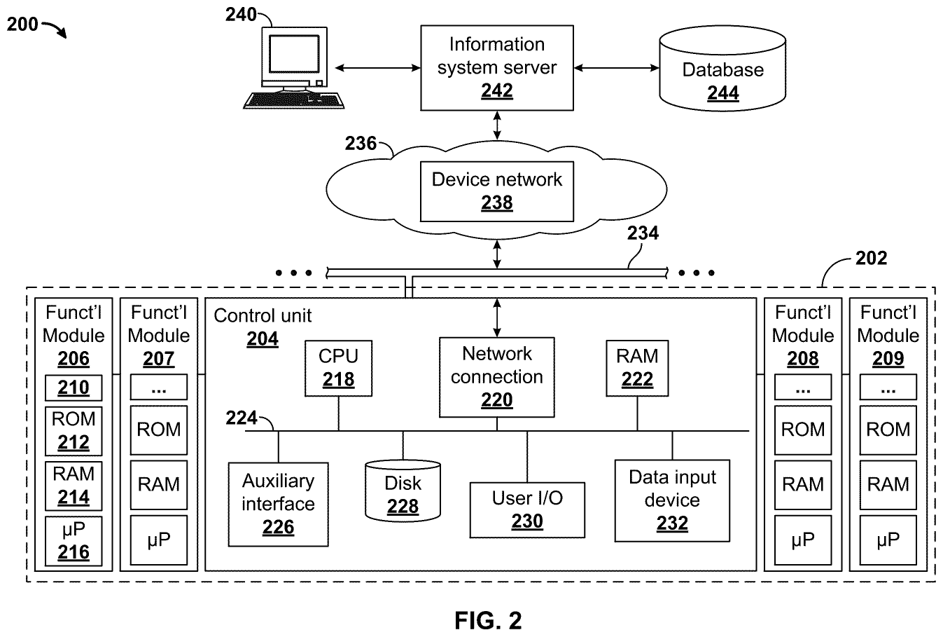

FIG. 2 depicts an example institutional patient care system 200 of a healthcare organization, according to various aspects of the subject technology. In FIG. 2, a patient care device 202 (e.g., patient care device 102 of FIGS. 1A and 1B) is connected to an internal healthcare network 236. The term patient care device (“PCD”) may be used interchangeably with the term patient care unit (“PCU”), either of which may include various ancillary medical devices, such as an infusion pump (e.g., infusion pumps 130-133 of FIG. 1A), a vital signs monitor, a medication dispensing device (e.g., cabinet, tote), a medication preparation device, an automated dispensing device, a module coupled with one of the aforementioned (e.g., a syringe pump module coupled with an infusion pump), and the like. Each element of the patient care device 202 is connected to an internal healthcare network 236 by a transmission channel 234. The transmission channel 234 can be a wired or wireless transmission channel, such as an 802.11 wireless local area network (LAN).

In some implementations, the internal healthcare network 236 also includes computer systems located in various departments throughout a hospital or healthcare center. For example, the internal healthcare network 236 optionally includes computer systems associated with an admissions department, a billing department, a biomedical engineering department, a clinical laboratory, a central supply department, one or more unit station computers, and/or a medical decision support system. As described further below, the internal healthcare network 236 may include discrete subnetworks. In the depicted example, the internal healthcare network 236 includes a device network 238 by which the patient care device 202 and other devices can communicate in accordance with normal operations.

The institutional patient care system 200 may also incorporate a separate information system server 242 (e.g., a health information system server). Moreover, although the information system server 242 is shown as a separate server, the functions and programming of the information system server 242 may be incorporated into another computer. The institutional patient care system 200 may further include a device terminal 240 for connecting and communicating with information system server 242. The device terminal 240 may include personal computers, personal data assistants, or mobile devices (e.g., laptops, tablet computers, augmented reality devices, or smartphones) configured with software for communications with information system server 242 via the internal healthcare network 236.

Patient care device 202 comprises a system for providing patient care, and it may include or incorporate infusion pumps (e.g., infusion pumps 130-133 of FIG. 1A), physiological monitors (e.g., heart rate, blood pressure, ECG, EEG, pulse oximeter, and other monitors), therapy devices, and other drug delivery devices that may be utilized according to the teachings set forth herein.

In the depicted example, patient care device 202 comprises a control unit 204 (e.g., control unit 104 of FIGS. 1A and 1B), also referred to as interface unit 204, connected to one or more functional modules 206-209 (e.g., infusion pumps 130-133 of FIG. 1A). Control unit 204 includes a central processing unit (CPU) 218 connected to a memory, for example, random access memory (RAM) 222, and one or more interface devices such as user interface device 230, a coded data input device 232, a network connection 220, and an auxiliary interface 226 for communicating with additional modules or devices. Control unit 204 also, although not necessarily, includes a main non-volatile storage unit 228, such as a hard disk drive or non-volatile flash memory, for storing software data. Additionally, control unit 204 may include one or more internal buses 224 for interconnecting the aforementioned elements.

In various implementations, user interface device 230 is a touch screen for displaying information to a user and allowing a user to input information by touching defined areas of the screen. Additionally, or in the alternative, user interface device 230 could include any means for displaying and inputting information, such as a monitor, a printer, a keyboard, softkeys, a mouse, a track ball, and/or a light pen.

Data input device 232 may be a bar code reader capable of scanning and interpreting data printed in bar coded format. Additionally, or in the alternative, data input device 232 can be any device for entering coded data into a computer, such as a device(s) for reading magnetic strips, radio-frequency identification (RFID) devices whereby digital data encoded in RFID tags or smart labels (defined below) are captured by the data input device 232 via radio waves, PCMCIA smart cards, radio frequency cards, memory sticks, CDs, DVDs, or any other analog or digital storage media. Other examples of the data input device 232 include a voice activation or recognition device or a portable personal data assistant (PDA). Depending upon the types of interface devices used, the user interface device 230 and the data input device 232 may be the same device. Although the data input device 232 is shown in FIG. 2 as being disposed within the control unit 204, the data input device 232 may be external to the control unit 204 (e.g., at the device terminal 240).

Auxiliary interface 226 may be an RS-232 communications interface, however any other means for communicating with a peripheral device (e.g., a printer, a patient monitor, an infusion pump, or another medical device) may be used without departing from the subject technology. Additionally, the data input device 232 may be a separate functional module (e.g., functional modules 206-207) configured to communicate with the control unit 204 or any other system on the network using suitable programming and communication protocols.

Network connection 220 may be a wired or wireless connection, such as by Ethernet, Wi-Fi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

The functional modules 206-209 are devices (e.g., infusion pumps 130-133 of FIG. 1A) for providing care to a patient or for monitoring patient conditions. As shown in FIG. 2, at least one of functional modules 206-209 may be an infusion pump module such as an intravenous infusion pump for delivering medication or other fluid to a patient. For the purposes of this discussion, functional module 206 is an infusion pump module. Each of functional modules 206-209 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Additionally, the functional modules 206-209 may include a printer, a scanner, a bar code reader, a near-field communication reader, an RFID reader, or any other peripheral input, output or input/output device.

Each functional module 206-209 communicates directly or indirectly with control unit 204, providing overall monitoring and control of the patient care device 202. Additionally, the functional modules 206-209 may be connected physically and electronically in serial fashion to one or both ends of control unit 204 as shown in FIG. 2. However, it is recognized that there are other means for connecting the functional modules 206-209 with the control unit 204 that may be utilized without departing from the subject technology. It is also appreciated that devices such as pumps or patient monitoring devices that provide sufficient programmability and connectivity may be capable of operating as stand-alone devices and may communicate directly with the internal healthcare network 236 without being connected through the control unit 204 or a separate interface unit. As described above, additional medical devices or peripheral devices may be connected to the patient care device 202 through one or more auxiliary interfaces 226.

Each of the functional modules 206-209 may include a microprocessor 216, a volatile memory 214, a nonvolatile memory 212, and module-specific components 210. It should be noted that while four functional modules are shown in FIG. 2, any number of devices may be connected directly or indirectly to the control unit 204. The number and type of functional modules described herein are intended to be illustrative, and they in no way limit the scope of the subject technology. The module-specific components 210 include any components necessary for operation of a particular module, such as a pumping mechanism for the functional module 206.

While each of the functional modules 206-209 may be capable of a least some level of independent operation, the control unit 204 monitors and controls overall operation of the patient care device 202. For example, as will be described in more detail below, the control unit 204 provides programming instructions to the functional modules 206-209 and monitors the status of each of the functional modules 206-209.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, the patient care device 202 and the internal healthcare network 236 may communicate via automated interaction, manual interaction, or a combination of both automated and manual interaction. Automated interaction may be continuous or intermittent and may occur through the network connection 220, as shown in FIG. 2, or through RS232 links, MIB systems, RF links such as BLUETOOTH, IR links, WLANS, digital cable systems, telephone modems, or other wired or wireless communication means.

Manual interaction between the patient care device 202 and the internal healthcare network 236 involves physically transferring, intermittently or periodically, data between systems using, for example, the user interface device 230, the coded data input device 232, bar codes, computer disks, portable data assistants, memory cards, or any other media for storing data. The communication means in various aspects is bidirectional with access to data from as many points of the distributed data sources as possible. Decision-making can occur at a variety of places within the internal healthcare network 236. For example, and not by way of limitation, decisions can be made in the information system server 242, decision support, a remote data server, hospital department or unit stations, or within the patient care device 202 itself.

According to various implementations, the information system server 242 includes a formulary and/or pharmacy information system. Pharmacy information systems may enable a safer physician medication order process. A pharmacy website (e.g., provided by the information system server 242) may provide the physician with a list of available drugs from which the physician may select. The pharmacy website may contain a drug library having the list of available drugs but may also contain and present to the physician the drug names associated with recommended dosages and dose limits that have been established or adopted by the healthcare facility. In such a case where the physician need only select items from the computer screen rather than having to manually type in drug names and drug administration numbers (such as infusion rates, times, etc.) associated with administration of the medication, a more accurate medication process should result.

If a clinical order is for administration of a particular medication regimen, the order will be transmitted to the pharmacy's system server (e.g., information system server 242). The pharmacy reviews the order, and once the order has been prepared, the order may be transmitted to the nurse station for matching with the appropriate patient. Within a formulary, there may be indication for use information and/or concentrations and drug ranges approved for the facility. A formulary is an approved list of drugs for use (e.g., available to order for a patient) within a medical facility. As will be described further, a formulary may be used to define one or more medical device drug libraries, which may then be provided to infusion pumps within a hospital network (e.g., internal healthcare network 236).

Inside the drug library, there is medication information such as drug names, concentration, diluent volume, strength, minimum or maximum infusion parameters for a drug, and other parameters. The establishment of these parameters, along with parameters for off-formulary orders, via the information pharmacy's system server is useful for maintaining consistency across the healthcare environment and ensuring an order is intelligible and executed according to expectations by other devices (e.g., patient care device 202) within the pharmacy's system server.

With further reference to FIG. 2, the patient care device 202 is capable of operating in several different modes, or personalities, with each personality defined by a configuration database. The configuration database may be a database internal to the patient care device 202, or an external database 244. A particular configuration database is selected based, at least in part, by patient-specific information such as patient location, age, physical characteristics, or medical characteristics.

Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or the location of the patient care device 202 location in the hospital or hospital computer network. Patient care information may be entered through any of the network connection 220, the user interface device 230, the data input device 232, or the auxiliary interface 226. Additionally, patient care information may originate from anywhere in the internal healthcare network 236, such as from a pharmacy server, an admissions server, a laboratory, and the like.

The memory of the control unit 204 (e.g., RAM 222 or the main non-volatile storage unit 228) may contain a drug library, an event log, and/or infusion pump configuration settings, such as profiles to be used in particular practice areas (e.g., ICU, PED, etc.). The control unit 204 memory may be electronically loadable memory such as non-volatile memory (e.g., EEPROM). Drug libraries stored on pumps, which illustratively contain such information as the drug names, ranges of delivery parameter values such as proper concentrations, dosage units, and dose limits, can be used to perform drug-calculation-based infusions in a clinical setting.

A drug library stored within the pump's memory may include clinical order settings such as limits set by the clinical institution for each drug of the library (also termed as “guardrails” herein). Such limits may take the form of maximum and minimum dosages for each drug which may be made dependent on patient factors or other factors associated with delivery of the drug. For example, the dosage limits may vary depending on the weight of the patient or body surface area (“BSA”), depending on the unit or ward of the medical institution in which the drug is being used (for example neonatal care unit (NCU), the intensive care unit (ICU), etc.), and/or other factors. The limits may also include maximum and minimum flow rates, infusion times, or other infusion parameters for a given drug. As with the dosage limits, these limits may also vary depending on the weight or other physiological parameters of the patient. In some implementations, the drug library and/or guardrails are stored elsewhere, such as on the device terminal 240, the external database 244, or another device connected to the internal healthcare network 236.

An alarm may be provided if the nurse sets the pump to operate outside the range between the limits for a particular drug. In some cases, the alarm may be overridden and in other cases it may not. The medical facility may establish “soft” limits for each drug, which may be overridden by the nurse, and “hard” limits which may not. In either case where a limit is exceeded, a pump data log or other processor in communication with the infusion pump may record each such limit event for later analysis where the attempted setting is higher than the maximum or lower than the minimum dosage.

The pump may also include a display (e.g., display 114 of FIG. 1B) for displaying a user interface, which may include a control panel through which the user can program the programmable controller and a display screen for displaying drug entries from the drug library. Each of the associated sets of drug delivery parameters includes information selected from a group of parameters including drug concentration, drug delivery rate, drug dose, and bolus size. The electronically loaded drug library contains a list of available mode options specifying the units available for expressing drug delivery information, and the drug infusion pump offers the user the list of available mode options from which to make a selection when the electronically loaded drug library is in the pump.

In the case of a syringe pump, the electronically loaded drug library may include a list of names of syringe manufacturers identifying syringes that can be used in the drug infusion pump, and the drug infusion pump offers the user the list of names of syringe manufacturers from which to make a selection when the electronically loaded drug library is in the pump. The loaded drug library may include a list of syringe sizes identifying syringes that can be used in the drug infusion pump, and the drug infusion pump offers the user the list of syringe sizes from which to make a selection when the electronically loaded drug library is in said pump. In the case of a peristaltic pump, the electronically loaded drug library may include a list of infusion set manufacturers. A loaded drug library may include a set of features, each of which is either be toggled on or off, and the pump offers the user only the features from among the set of features that are toggled on when the electronically loaded drug library is in said pump.

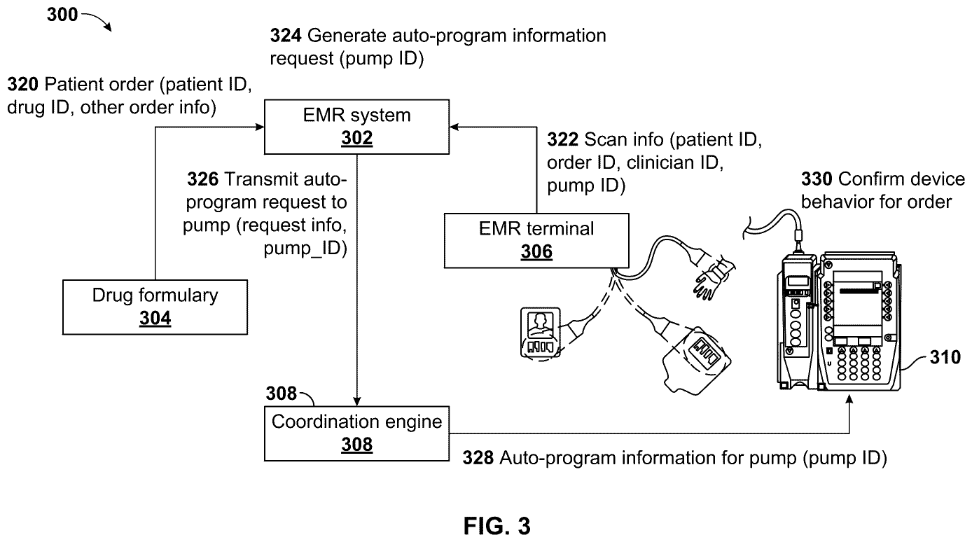

FIG. 3 depicts an example system 300 for automatically programming a medical device (e.g., using a supplemented APR), according to various aspects of the subject technology. Interoperability between a hospital EMR server (e.g., information system server 242 of FIG. 2) and medical devices (e.g., patient care device 102 of FIGS. 1A-B or patient care device 202 of FIG. 2) enable pre-population of infusion parameters. Pre-population of infusion parameters may reduce the number of programming screens and key presses required with manually programming a pump. The implementation of interoperability does not preclude a clinician from manually programming the infusion device. Manual programming may be required in the event of a failure in any component of the interfaced system.

While features may be described with reference to an EMR server, the features are applicable to provide auto-programming of medical devices using similar hospital information systems such as patient data management systems (PDMS). Furthermore, while the features may be described using an infusion pump as the example medical device, the features are applicable to provide auto-programming of other medical devices using barcodes for association such as patient monitors, patient association management systems, or alarms management systems.

A drug formulary 304 determines which medications can be dispensed within a hospital network (e.g., internal healthcare network 236 of FIG. 2). A hospital committee may be formed to determine how medications within that formulary would be applied to an infusion device 310 (e.g., patient care device 102 of FIGS. 1A and 1B or patient care device 202 of FIG. 2). Configuration definitions (e.g., by a hospital unit such as ICU, NICU, Pediatrics, Oncology, Surgery, etc.) are agreed upon and the drugs and typical infusion protocols are established in a medical device drug library.

In addition, limitation conditions, or “guardrail” conditions, may be defined in the drug library. When the definitions are complete, a configuration can then be released including the drug library. Infusion devices at the institution can then be updated by transferring the configuration databases into some or all of their pumps. Corresponding updates to the drug formulary 304 may be shared with other hospital systems such as the pharmacy ordering system or an EMR system 302 which may use formulary information to generate a patient order to deliver a particular drug to a particular patient (320).

In the clinical field, a clinician may scan a medical item such as an infusate package using a scanner associated with a medical device, such as the infusion device 310. For example, a bar code reader (or other data input device) is used to scan the coded drug label, the patient's coded ID band and the caregiver's ID badge, and optionally supplementary prescription information or medical device configuration instructions (including configuration database ID) printed on the label or an accompanying order.

The reader/scanner is not required to be integrated with a medical device. The scanner may be part of a separate device, such as an EMR terminal 306 (e.g., device terminal 240 of FIG. 2), that is connected to the same network as the infusion device 310 (e.g., device network 238 of FIG. 2) and configured with software to function in an overall workflow involving the infusion device 310.

The scanning initiates a process by which information pertaining to the item (e.g., scanned from a code affixed to or transmitted by the item) is automatically sent to the EMR system 302 via a network (e.g., internal healthcare network 236) (322). The EMR system 302 may confirm the item and generate (324) and send an automated programming request (APR) to the infusion device 310 to load parameters pertaining to the item. The parameters may be stored in the infusion device 310, but loaded in response to an identifier received from the server. While the examples herein involve an infusion device, any medical device may be configured in the same or similar manner and employ the automated programming error mitigation described herein.

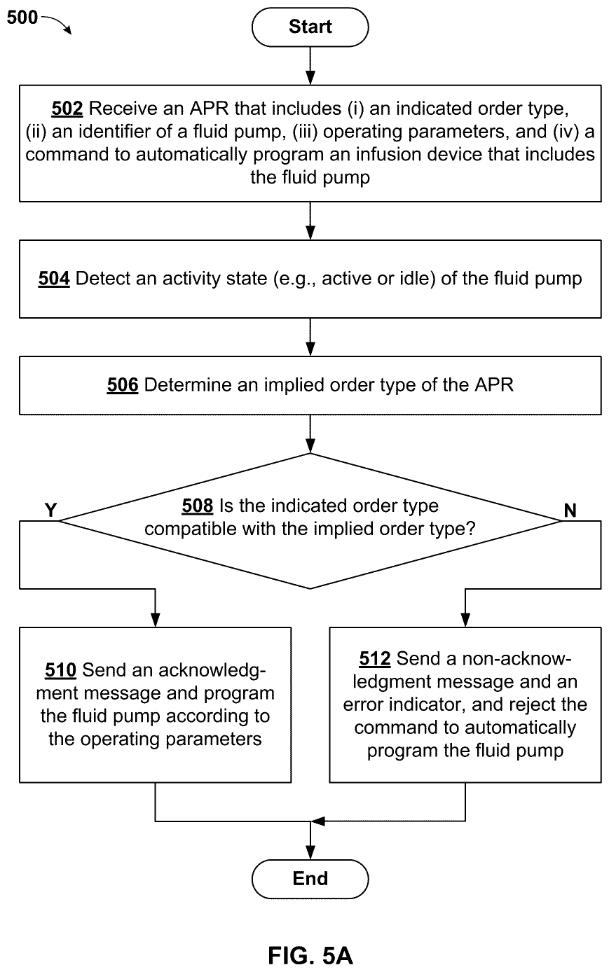

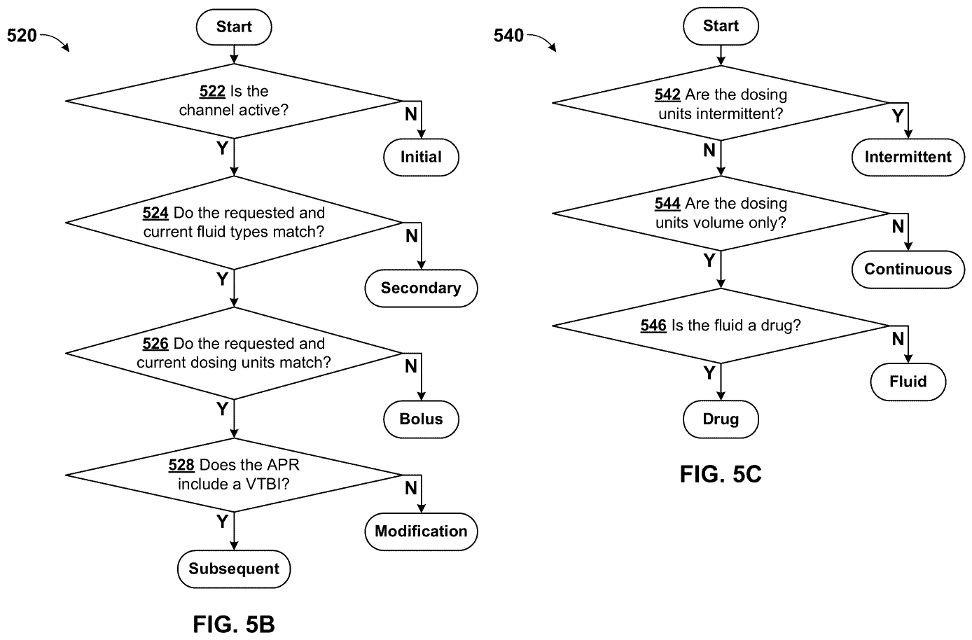

In the depicted implementation, a coordination engine 308 coordinates messages sent from the EMR system 302 to the infusion device 310. In the depicted implementation, the EMR system 302 transmits an APR to the coordination engine 308 with a device identifier (326), also known as a “device ID,” of the infusion device 310 to receive the APR. The coordination engine 308 then determines whether the infusion device 310 identified by the EMR system 302 is available and, if so, forwards the APR to the infusion device 310 (328). When an APR received by the infusion device 310, the infusion device 310 programs itself according to the parameters of (or associated with) the APR. The coordination engine 308 may also supplement the APR prior to sending it to the infusion device 310, as discussed in more detail below with respect to FIG. 5.

In some implementations, the APR activates a drug library stored on the infusion device 310, and the infusion device 310 is programmed according to parameters stored in the drug library for medication identified in the APR. In some implementations, upon successful programming, the infusion device may automatically self-configure and, in some instances, initiate operation based on the parameters. In some implementations, the infusion device 310 may confirm the automatically entered parameters (330). The confirmation may include presenting one or more user interface screens including the parameters and values along with a control element (e.g., a button) that, when activated, causes the infusion device to begin operation based on the parameters. The user interface may include additional or alternative control elements to allow a clinician to adjust an automatically entered parameter based on, for example, professional judgement or changes in patient condition.

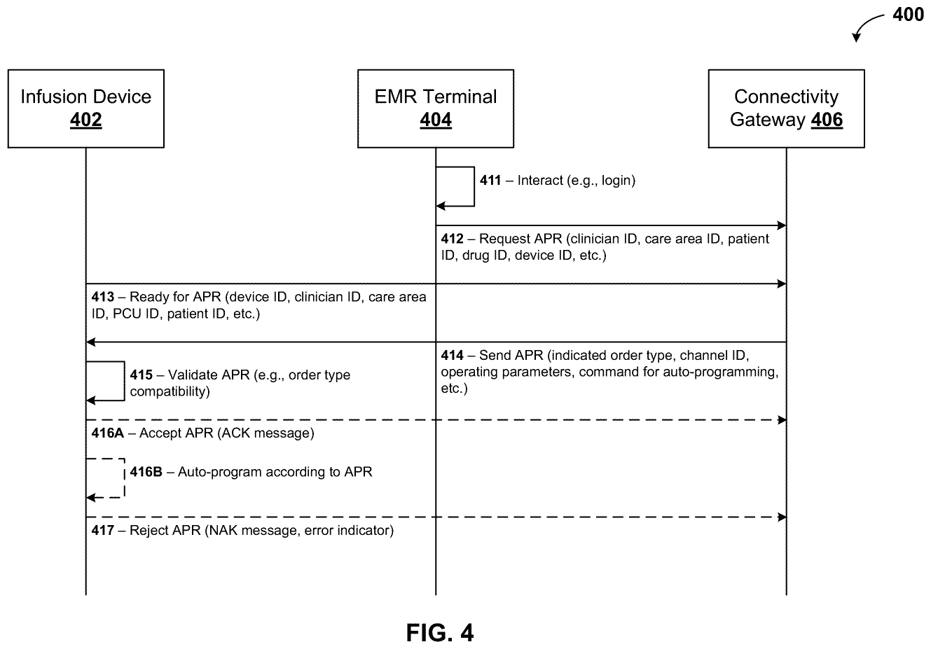

FIG. 4 depicts an example sequence diagram for automatically validating an APR, according to various aspects of the subject technology. For explanatory purposes, the various blocks of the example sequence diagram 400 are described with reference to FIGS. 1A through 3 and the components and/or processes described therein.

As discussed above, an identifier (e.g., a bar code) of an infusion device 402 (e.g., patient care device 102 of FIGS. 1A and 1B, patient care device 202 of FIG. 2, or infusion device 310 of FIG. 3) may be scanned at an EMR terminal 404 (e.g., device terminal 240 of FIG. 2 or EMR terminal 306 of FIG. 3). This may initiate a process whereby information pertaining to the infusion device 402 is automatically sent to a connectivity gateway 406, as illustrated in FIG. 4. The connectivity gateway 406 (or a server performing a similar function) can perform certain actions pertaining to the infusion device 402 and ultimately cause transmission of an APR to the infusion device 402. After receiving the APR, the infusion device 402 can then validate the APR and respond accordingly. In some implementations, an identifier of the infusion device 402 is scanned from a barcode affixed to the device in conjunction with parameters entered into the EMR terminal 404 for selecting and/or generating the APR.

A hospital system implementing the subject technology may include the aforenoted infusion device 402, EMR terminal 404, and connectivity gateway 406. In some implementations, the connectivity gateway 406 may include one or more computing devices, such as a server (e.g., an EMR server such as EMR system 302 of FIG. 3). In some implementations, the connectivity gateway 406 is implemented by, or interchangeable with, the coordination engine 308 of FIG. 3. Moreover, in some implementations, the information system server 242 of FIG. 2 is representative of the connectivity gateway 406.

In the depicted example of FIG. 4, a clinician interacts (411) with the EMR terminal 404. This interaction may include, for example, the clinician scanning a badge or authentication device associated with the clinician. As described with regard to FIG. 3, the EMR terminal 404 can obtain the clinician's identification by way of the authentication process. Additionally, the clinician may enter a care area identifier, a patient identifier, a drug identifier, an identifier of the infusion device 402, and/or other identifying information for requesting that an APR be sent to the infusion device 402. After the clinician interacts with the EMR terminal 404, the EMR terminal then transmits (412) an APR request to the connectivity gateway 406.

Separately, in some implementations, the infusion device 402 (e.g., via a control unit such as control unit 104 of FIGS. 1A and 1B) sends (413) a message to the connectivity gateway 406 indicating that the infusion device 402 is ready to receive automated programming. This message may include, for example, parameters such as an identifier of the infusion device 402 (e.g., a serial number), an identifier of a clinician assigned to the infusion device 402 (e.g., a clinician logged into the device), an identifier of the care area in which the infusion device 402 is located, an identifier of a control unit (e.g., control unit 104 of FIGS. 1A and 1B or control unit 204 of FIG. 2) of the infusion device 402, an identifier of a patient associated with the infusion device 402, and/or other identifying information necessary for the connectivity gateway 406 to send an APR to the infusion device 402.

After receiving the APR request from the EMR terminal 412 (and/or, in some implementations, the indication from the device 402), the connectivity gateway 406 sends (414) the APR to the infusion device 402. The APR may include, for example, an indicated order type that denotes a type of an order associated with the APR (e.g., an initial order, a subsequent order, a modification order, a bolus order, or a secondary order). The APR may also include an identifier (e.g., a channel indicator) of a fluid pump (e.g., any of infusion pumps 130-133 of FIG. 1A) of the infusion device 402. Additionally, the APR may include operating parameters for administering the order, such as a fluid type, dosing units (e.g., a dosage amount), a flow rate, and/or a volume-to-be-infused (VTBI). Further, the APR may include a command to automatically program the infusion device 402 according to the operating parameters.

According to various implementations described herein, the infusion device 402 attempts to validate (415) the APR after receiving it. In some implementations, the APR validation process includes a “fail-fast” process that attempts to identify high-level errors (e.g., compatibility issues between the indicated and implied order types of the APR) before determining whether the APR includes more difficult-to-detect errors. In this manner, the APR validation process may quickly determine whether the APR is faulty and/or should be rejected. Example APR validation processes, including fast fail processes, are discussed in more detail below with respect to FIGS. 5A through 5C.

If the infusion device 402 successfully validates the APR, then the infusion device 402 accepts (416A) the APR. This may include, for example, transmitting an acknowledgement message, or “ACK” message, to the connectivity gateway 406. Additionally, after validating the APR, the infusion device 402 may automatically program (416B) itself according to the APR (e.g., according to the operating parameters). On the other hand, if the infusion device 402 cannot validate the APR (e.g., due to an error with the APR), then the infusion device 402 rejects (417) the APR, for example, by transmitting a non-acknowledgement message, or “NAK” message, and/or an error message that indicates any errors identified with the APR during the validation process. The non-acknowledgement and/or error message(s) may be sent to the connectivity gateway 406, which may forward the message(s) to another device (e.g., the EMR server, EMR terminal, or a computing device associated with the requesting clinician).

FIG. 5A depicts an example process 500 for validating APRs, according to various aspects of the subject technology. For explanatory purposes, the present disclosure describes the blocks of example process 500 with reference to FIGS. 1A-4, including the components and/or processes described therein. One or more of the blocks of process 500 may be implemented by one or more of the computing devices described therein, such as the patient care device 102 of FIGS. 1A and 1B, the patient care device 202 of FIG. 2, the infusion device 310 of FIG. 3, and/or the infusion device 402 of FIG. 4.

In some implementations, one or more of the blocks may be implemented based on one or more machine-learning algorithms. In some implementations, one or more of the blocks may be implemented apart from other blocks, and by one or more different processors or devices. Further, for explanatory purposes, the blocks of the process 500 are described as occurring in serial, or linearly. However, multiple blocks of the process 500 may occur in parallel. Additionally, the blocks of the process 500 need not be performed in the order shown and one or more of the blocks of the process 500 need not be performed whatsoever.

In the depicted example, a processor of an electronic device (e.g., patient care device 102 of FIGS. 1A and 1B, patient care device 202 of FIG. 2, infusion device 310 of FIG. 3, or infusion device 402 of FIG. 4) receives (502) an APR from a device manager (e.g., connectivity gateway 406 of FIG. 4) (see also 414 of FIG. 4). The APR includes an indicated order type (e.g., an initial order, a subsequent order, a modification order, a bolus order, or a secondary order), an identifier of a fluid pump (e.g., a channel indicator designating the fluid pump) of the infusion device (e.g., fluid pumps 130-133 of FIG. 1A), operating parameters, and a command to automatically program an infusion device according to the operating parameters.

The indicated order type for the APR may be selected by a clinician, for example, when the clinician requests automated programming of the infusion device at an EMR terminal (e.g., EMR terminal 404 of FIG. 4) (see 411 of FIG. 4). In this manner, the indicated order type may suggest that the clinician intends for the APR to result in the administration of a particular type of order via the infusion device. In some implementations, order types include initial orders, subsequent orders, modification orders, bolus orders, and secondary orders.

Moreover, the operating parameters of the APR may include, for example, a requested fluid type for administration via the fluid pump. The operating parameters may also include requested dosing units (e.g., a requested dosage amount) for operating the fluid pump. Additionally, the operating parameters may include a VTBI, a flow rate, and/or other parameters for administering an infusion therapy via the fluid pump.

The processor also detects (504) an activity state of the fluid pump. The activity state may be, for example, an idle state that indicates the fluid pump is not pumping a fluid or an active state that indicates that the fluid pump is pumping a fluid. Additionally, the processor determines (506) an implied order type for the APR based in part on the detected activity state. As with the indicated order type for the APR, in some implementations, the implied order type may include an initial order, a subsequent order, a modification order, a bolus order, or a secondary order. Unlike the indicated order type, however, the implied order type may not have been selected by a clinician or otherwise indicated by a user. Rather, the implied order type may include an order type suggested by the operating parameters of the APR, as well as other contextual parameters suggestive of an order type to be administered in accordance with the APR. For further discussion of this determination, see FIG. 5B and the corresponding disclosure below.

After determining the implied order type, the processor determines (508) whether the indicated order type is compatible with the implied order type (and, in some implementations, the fluid pump) (see 415 of FIG. 4). This determination may be based, for example, on whether the indicated order type matches the implied order type, as well as, in some implementations, an activity state of the target fluid pump and/or a type of an active infusion being administered by the target fluid pump. If the order type is compatible with the implied order type (and the fluid pump), the processor sends (510) an acknowledgment message to the device manager (see 416A of FIG. 4) and programs the fluid pump according to the operating parameters (see 416B of FIG. 4). However, if the order type is incompatible with the implied order type (or the fluid pump), the processor sends (512) a non-acknowledgment message and/or an error message (see 417 of FIG. 4) and rejects the command to automatically program the fluid pump.

The processor may also determine an active infusion type and use the active infusion type in determining whether the indicated order type is compatible with the implied order type and the fluid pump. For example, the processor may determine, responsive to detecting that the activity state includes the active state, an active infusion type of the fluid pump based in part on active dosing units (e.g., an active dosage amount) or an active fluid type. The active infusion type denotes a type of an infusion therapy actively being administered by the infusion device via the fluid pump. Potential infusion types include fluid infusions, drug infusions, continuous infusions, and intermittent infusions. The determination of the active infusion type is discussed in more detail below with respect to FIG. 5C.

Determining whether the indicated order type and the implied order type are compatible may include various determinations based in part on whether the indicated order type matches the implied order type and/or based on the active infusion type of the fluid pump. In some implementations, determining whether the indicated order type is compatible with the implied order type and the fluid pump includes, responsive to determining that the implied order type is the initial order: (i) responsive to determining that the indicated order type is the initial order or the subsequent order, determining that the indicated order type is compatible with the implied order type and the fluid pump, and (ii) responsive to determining that the indicated order type is the modification order, the bolus order, or the secondary order, determining that the indicated order type is incompatible with the implied order type or the fluid pump.

In some implementations, determining whether the indicated order type is compatible with the implied order type and the fluid pump includes, responsive to determining that the implied order type is the subsequent order: (i) responsive to determining that the indicated order type is the initial order or the subsequent order, determining that the indicated order type is compatible with the implied order type and the fluid pump, and (ii) responsive to determining that the indicated order type is the modification order, the bolus order, or the secondary order, determining that the indicated order type is incompatible with the implied order type or the fluid pump.

In some implementations, determining whether the indicated order type is compatible with the implied order type and the fluid pump includes, responsive to determining that the implied order type is the modification order: (i) responsive to determining that the indicated order type is the modification order, determining that the indicated order type is compatible with the implied order type and the fluid pump; and responsive to determining that the indicated order type is the initial order, the subsequent order, the bolus order, or the secondary order, determining that the indicated order type is incompatible with the implied order type or the fluid pump.

In some implementations, determining whether the indicated order type is compatible with the implied order type and the fluid pump includes, responsive to determining that the implied order type is the bolus order: (i) responsive to determining that the indicated order type is the bolus order: (a) determining whether the fluid pump is configured to support the bolus order, (b) responsive to determining that the fluid pump is configured to support the bolus order, determining that the indicated order type is compatible with the implied order type and the fluid pump, and (c) responsive to determining that the fluid pump is not configured to support the bolus order, determining that the indicated order type is incompatible with the implied order type or the fluid pump, and (ii) responsive to determining that the indicated order type is the initial order, the subsequent order, the modification order, or the secondary order, determining that the indicated order type is incompatible with the implied order type or the fluid pump.

In some implementations, the comparison between the indicated order type and the implied order type of an APR is used during manufacturing and calibration of an infusion device. For example, an APR may be provided to the infusion device with an indicated order type that is incompatible with an implied order type of the APR (e.g., based on the operating parameters thereof). Based on whether and/or how the infusion device rejects the APR (e.g., returning a message indicating compatibility issues with the APR), the infusion device may be deemed to be properly calibrated for APR validation. The calibration process may also include providing an APR to the infusion device, where the indicated and implied order types of the APR are not the same but are compatible and/or are the same and are compatible.

In some implementations, determining whether the indicated order type is compatible with the implied order type and the fluid pump includes, responsive to determining that the implied order type is the secondary order: (i) responsive to determining that the indicated order type is the secondary order: (a) determining whether the fluid pump is configured to support the secondary order, (b) responsive to determining that the fluid pump is configured to support the secondary order, determining that the indicated order type is compatible with the implied order type and the fluid pump, and (c) responsive to determining that the fluid pump is not configured to support the secondary order, determining that the indicated order type is incompatible with the implied order type or the fluid pump, and (ii) responsive to determining that the indicated order type is the initial order, the subsequent order, the modification order, or the bolus order, determining that the indicated order type is incompatible with the implied order type.

The processor may also display a notice via a display device (e.g., display 114 of FIG. 1B or user interface device 230 of FIG. 2) associated with the infusion device. The notice may include information, for example, regarding the validation of the APR. If the APR is validated successfully, then the information may indicate successful validation. However, if the APR cannot be validated, then the information may indicate one or more reasons for the APR failing validation (e.g., due to incompatibility between (i) the indicated order type and (ii) the implied order type and/or the fluid pump.

For example, the processor may, responsive to determining that the indicated order type is incompatible with the implied order type or the fluid pump determine that the operating parameters of the APR are incompatible with the fluid pump or determine that the implied order type is incompatible with an active infusion type of the fluid pump. Accordingly, the processor may display a first notice via the display device, where the first notice indicates, for example, that the APR is rejected and that the operating parameters of the APR are incompatible with the fluid pump. Or the processor may display a second notice via the display device, where the second notice indicates that the APR is rejected, that the implied order type is incompatible with the active infusion type of the fluid pump, and the active infusion type of the fluid pump. The processor may also display alternative or additional information based on an error message also sent to the device manager. For example, if the implied order type is the bolus order, the indicated order type is the bolus order, and the active fluid pump does not support bolus steps, the processor may display information via the display device that indicates that the operating parameters of the APR are not supported for the fluid pump. The processor may also display the error message and/or an error indicator contained therein.

In some implementations, the processor is configured to display information indicating, for example, (i) that the APR was rejected, for instance, at a target fluid pump designated by the APR, (ii) that operating parameters or other parameters included in the APR are not supported for the target fluid pump, (iii) that the target fluid pump is busy and therefore cannot be programmed according to the APR (e.g., including an estimate of a time at which the target fluid pump will be ready and/or including an indication of the infusion type currently being administered by the target fluid pump), (iv) that the order type of the APR (e.g., an indicated order type and/or an implied order type) does not match a current fluid type of the fluid pump, (v) that the APR is missing pertinent information (e.g., necessary operating parameters), and/or (vi) that the APR includes invalid information.

The error message that the processor sends to the device manager responsive to determining that the indicated order type is incompatible with the implied order type or the fluid pump may be based in part, for example, on determinations made during the compatibility determination. The error message may include a program session identifier and/or a revision identifier for another channel (e.g., fluid pump) that matches the infusible (e.g., that is compatible with the APR).

The error message may also include an error indicator that denotes a type of the error encountered. For example, if the implied order type is the initial order and the indicated order type is the modification order, the error message may include a titrate-idle-channel error indicator. As another example, if the implied order type is the initial order and the indicated order type is the bolus order, the error message may include a bolus-idle-channel error indicator. As an additional example, if the implied order type is the initial order and the indicated order type is the secondary order, the error message may include a secondary-idle-channel error indicator. As yet another example, if the implied order type is the subsequent, modification, or bolus order, the indicated order type is the bolus or secondary order, and a bolus, secondary, or basic is currently active on the infusion device, the error message may include, respectively, a bolus-, secondary-, or basic-executing error indicator. As a yet further example, if the implied order type is the secondary, modification, or bolus order, the indicated order type is the bolus or secondary order, and nether a bolus, secondary, nor basic is currently active on the infusion device, the error message may include a channel-program-mismatch error indicator.

As an additional example, if the implied order type is the secondary order and the indicated order type is the modification order, the error message may include an invalid-information error indicator. As a further example, if the implied order type is the modification order and the indicated order type is the initial or subsequent order, the error message may include an invalid-information error indicator (e.g., as well as an identifier of any offending elements). As a further example, if the implied order type is the bolus order and the designated fluid channel (e.g., fluid pump) does not support bolus steps, the error message may include a bolus-unsupported error indicator. As another example, if the implied order type is the secondary order and the designated fluid channel (e.g., fluid pump) does not support secondary steps, the error message may include a secondary-unsupported error indicator. As yet another example, if the implied order type is the secondary order, the indicated order type is the initial, subsequent, modification, or bolus order, and a bolus, secondary, or basic is currently active on the infusion device, the error message may include, respectively, a bolus-, secondary-, or basic-executing error indicator. As a further example, if the implied order type is the secondary order, the indicated order type is the initial, subsequent, modification, or bolus order, and neither a bolus, secondary, or basic is currently active on the infusion device, the error message may include a channel-program-mismatch error indicator.

FIGS. 5B and 5C depict example processes 520 and 540 for determining, respectively, an implied order type of an APR and an active infusion type of a fluid pump, according to various aspects of the subject technology. As with the example process discussed above, one or more of the blocks of processes 520 and 540 may be implemented by one or more of the computing devices described herein, such as the patient care device 102 of FIGS. 1A and 1B, the patient care device 202 of FIG. 2, the infusion device 310 of FIG. 3, and/or the infusion device 402 of FIG. 4. Additionally, the blocks of the processes 520 and 540 need not be performed in the order shown and one or more of the blocks of the processes 520 and 540 need not be performed whatsoever.

As illustrated in FIG. 5B, the implied order type determination may be based on a variety of factors, including, for example, whether a target channel (e.g., a target fluid pump) is active, whether a requested fluid type and an active fluid type match, whether requested dosing units (e.g., a requested dosage amount) and active dosing units (e.g., an active dosage amount) match, and/or whether the APR includes a VTBI.

For example, in the illustrated implementation, the process 520 of determining an implied order type of an APR includes determining (522) (e.g., detecting) whether a channel designated by the APR (e.g., infusion pumps 130-133 of FIG. 1A) is active (e.g., in an active state). If the channel is inactive (e.g., in an idle state), then the processor may determine that the implied order type is an initial order. Additionally, the process 520 may include determining (524), responsive to detecting that the channel is active, whether a requested type of a fluid (e.g., as requested in the APR) matches that of a fluid actively being pumped by the channel. If the requested fluid type does not match the active fluid type, then the processor may determine that the implied order type is a secondary order. Moreover, the process 520 may include determining (526), responsive to determining that the requested fluid type matches the active fluid type, whether requested dosing units (e.g., as requested in the APR) match active dosing units (e.g., milliliters per hour, or milligrams) of the channel. In some implementations, determining whether the requested dosing units match the active dosing units comprises determining whether a type of the requested dosing units matches a type of the active dosing units. Example types of dosing units include intermittent units (e.g., specifying an amount of an active ingredient), continuous units (e.g., specifying an amount of time and an amount of an active ingredient), drug units (e.g., specifying a volume of a drug such as an analgesic), and fluid units (e.g., specifying a volume of a fluid such as saline).

If the requested dosing units do not match the active dosing units, then the processor may determine that the implied order type is a bolus order. Additionally, the process 520 may include determining (528), responsive to determining that the requested dosing units match the active dosing units, whether the operating parameters of the APR also include a requested VTBI (e.g., as requested in the APR). If the operating parameters do not include a requested VTBI, then the processor may determine that the implied order type is a modification order. However, if the operating parameters do include a requested VTBI, the processor may determine that the implied order type is a subsequent order.

Referring now to FIG. 5C, the example process 540 of determining an active infusion type of a fluid pump (e.g., infusion pumps 130-133 of FIG. 1A) includes determining (542) whether active dosing units of the fluid pump are intermittent units. As used herein, “intermittent units” refers to dosing units that include an amount (e.g., in milligrams) of an active ingredient but do not specify an amount of time (e.g., in seconds, minutes, or hours). Similarly, an intermittent infusion is an infusion of an amount of an active ingredient (e.g., a bolus), for example, over a small amount of time. If the active dosing units are intermittent units, then the processor may determine that the active infusion type is an intermittent infusion.

Responsive to determining that the active dosing units are not intermittent units, the process 520 also includes determining (544) whether the active dosing units consist of volume-only units (e.g., mL). If the active dosing units do not consist of volume-only units, then the processor may determine that the active infusion type is a continuous infusion. As used herein, “continuous infusion” describes a type of an infusion where an amount of an active ingredient (e.g., in milligrams) is administered over a particular amount of time. The process 520 may also include determining (546), responsive to determining that the active dosing units consist of volume-only units, whether the active fluid being pumped by the fluid pump is a drug (e.g., an analgesic or anesthetic). If the active fluid is not a drug (e.g., saline), then the processor may determine that the active infusion type is a fluid infusion. However, if the active fluid is a drug, then the processor may determine that the active infusion type is a drug infusion.

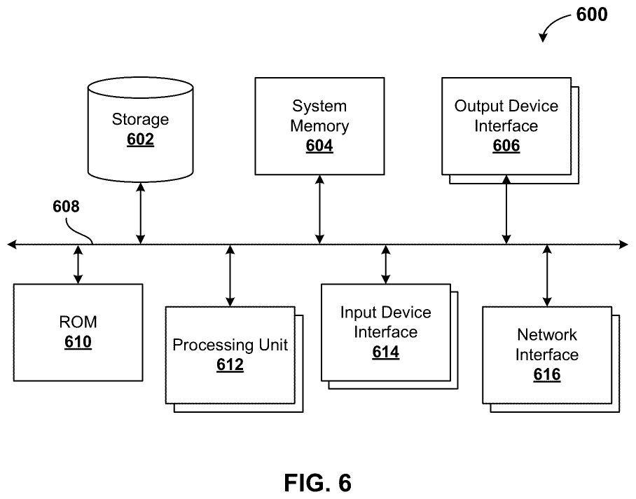

FIG. 6 is a conceptual diagram illustrating an example electronic system 600 for validating APRs, according to various aspects of the subject technology. The electronic system 600 may be implemented by a computing device for execution of software associated with portions or steps of process 600 of FIG. 6, or components and methods provided by FIGS. 1-5. In this regard, the electronic system 600 may include the patient care device 102 of FIGS. 1A and 1B, the patient care device 202 of FIG. 2, the infusion device 310 of FIG. 3, and/or the infusion device 402 of FIG. 4.

The electronic system 600 may also include a specifically-configured personal computer or a mobile device for infusion, such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Additionally, the electronic system 600 may include various types of computer-readable media and interfaces for various other types of computer-readable media. In the depicted example, the electronic system 600 includes a bus 608, a processing unit 612, a system memory 604, a read-only memory (ROM) 610, a permanent storage device 602, an input device interface 614, an output device interface 606, and a network interface 616. In some implementations, the electronic system 600 may include or be integrated with other computing devices or circuitry for operation of the various components and methods previously described.

The bus 608 collectively represents system, peripheral, and chipset buses that communicatively connect the numerous internal devices of the electronic system 600. For instance, the bus 608 communicatively connects processing unit 612 with the ROM 610, the system memory 604, and the permanent storage device 602. From these various memory units, the processing unit 612 retrieves instructions to execute and data to process in order to execute the processes of the subject disclosure. The processing unit 612 can be a single processor or a multi-core processor in different implementations.

The ROM 610 stores static data and instructions that are needed by processing unit 612 and other modules of the electronic system. The permanent storage device 602, on the other hand, is a read-and-write memory device. This device is a non-volatile memory unit that stores instructions and data even when the electronic system 600 is powered off. Some implementations of the subject disclosure use a mass-storage device (such as a magnetic or optical disk and its corresponding disk drive) as the permanent storage device 602. Other implementations use a removable storage device (such as a floppy disk, flash drive, and its corresponding disk drive) as the permanent storage device 602.

Like the permanent storage device 602, the system memory 604 is a read-and-write memory device. However, unlike the storage device 602, the system memory 604 is a volatile read-and-write memory, such as random-access memory (RAM). The system memory 604 stores some of the instructions and data that the processor needs at runtime. In some implementations, the processes of the subject disclosure are stored in the system memory 604, the permanent storage device 602, and/or the ROM 610. From these various memory units, the processing unit 612 retrieves instructions to execute and data to process, in order to execute the processes of some implementations.

The bus 608 also connects to the input device interface 614 and the output device interface 606. The input device interface 614 enables the user to communicate information and select commands to the electronic system. Input devices used with the input device interface 614 include, for example, alphanumeric keyboards and pointing devices (also called “cursor control devices”). The output device interface 606 enables, for example, the display of images generated by the electronic system 600. Output devices used with the output device interface 606 include, for example, printers and display devices, such as cathode ray tubes (CRT) or liquid crystal displays (LCD). Some implementations include devices (e.g., touchscreens) that function as both input and output devices.

Furthermore, the bus 608 also couples the electronic system 600 to a network (not shown) through the network interface 616. The network interface 616 may include, for example, a wireless access point (e.g., Bluetooth or Wi-Fi) or radio circuitry for connecting to a wireless access point. The network interface 616 may also include hardware (e.g., ethernet hardware) for connecting the computer to a part of a network of computers such as a local area network (LAN), a wide area network (WAN), wireless LAN, an intranet, or a network of networks, such as the Internet. Components of the electronic system 600 can be used in conjunction with the subject disclosure when specifically configured with one of more of the features described.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer-readable medium” and “computer-readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a LAN and a WAN, an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. An infusion device comprising: a fluid pump; and a processor configured to: receive an automated programming request (APR) from a device manager, the APR comprising (i) an indicated order type, (ii) an identifier of the fluid pump, (iii) operating parameters including a requested fluid type and a requested dosage amount, and (iv) a command to automatically program the infusion device according to the operating parameters; detect an activity state of the fluid pump, the activity state comprising (i) an idle state that indicates the fluid pump is not pumping a fluid or (ii) an active state that indicates the fluid pump is pumping a fluid; determine an implied order type for the APR based in part on the detected activity state, wherein the implied order type comprises an initial order, a subsequent order, a modification order, a bolus order, or a secondary order; determine whether the indicated order type is compatible with the implied order type and the fluid pump based in part on whether the indicated order type matches the implied order type; responsive to determining that the indicated order type is compatible with the implied order type and the fluid pump, (i) send an acknowledgment message to the device manager and (ii) program the fluid pump according to the operating parameters; and responsive to determining that the indicated order type is incompatible with the implied order type or the fluid pump, (i) send an error message to the device manager, and (ii) reject the command to automatically program the fluid pump according to the operating parameters.
Clause 2. The infusion device of Clause 1, wherein the operating parameters further comprise requested dosing units, and determining the implied order type comprises: determining, responsive to detecting that the activity state of the fluid pump comprises the idle state, that the implied order type comprises the initial order; determining, responsive to detecting that the activity state comprises the active state, whether the requested fluid type matches an active fluid type being pumped by the fluid pump; determining, responsive to determining that the requested fluid type does not match the active fluid type, that the implied order type comprises the secondary order; determining, responsive to determining that the requested fluid type matches the active fluid type, whether the requested dosing units match active dosing units of the fluid pump; determining, responsive to determining that the requested dosing units do not match the active dosing units, that the implied order type comprises the bolus order; determining, responsive to determining that the requested dosing units match the active dosing units, whether the operating parameters of the APR also include a requested VTBI; determining, responsive to determining that the operating parameters do not include the requested VTBI, that the implied order type comprises the modification order; and determining, responsive to determining that the operating parameters include the requested VTBI, that the implied order type comprises the subsequent order.
Clause 3. The infusion device of either of Clauses 1 or 2, wherein: the processor is further configured to, responsive to detecting that the activity state comprises the active state, determine an active infusion type of the fluid pump based in part on active dosing units or an active fluid type, the active infusion type comprising a fluid infusion, a continuous infusion, or an intermittent infusion; and determining whether the indicated order type is compatible with the implied order type and the fluid pump is further based on the active infusion type.
Clause 4. The infusion device of Clause 3, wherein determining the active infusion type comprises: determining whether active dosing units of the fluid pump comprise intermittent units; responsive to determining that the active dosing units comprise the intermittent units, determining that the active infusion type comprises the intermittent infusion; responsive to determining that the active dosing units do not comprise the intermittent units: determining whether the active dosing units consist of volume units; responsive to determining that the active dosing units do not consist of the volume units, determining that the active infusion type comprises the continuous infusion; and responsive to determining that the active dosing units consist of the volume units, determining whether the active fluid being pumped by the fluid pump is a drug, wherein the active infusion type comprises the fluid infusion when the active fluid is not a drug, and the active infusion type comprises the drug infusion when the active fluid is a drug.
Clause 5. The infusion device of any one of Clauses 1 through 4, wherein determining whether the indicated order type is compatible with the implied order type and the fluid pump comprises, responsive to determining that the implied order type comprises an initial order: determining, responsive to determining that the indicated order type comprises the initial order or the subsequent order, that the indicated order type is compatible with the implied order type and the fluid pump; and determining, responsive to determining that the indicated order type comprises the modification order, the bolus order, or the secondary order, that the indicated order type is incompatible with the implied order type or the fluid pump.
Clause 6. The infusion device of either of Clauses 3 or 4, wherein determining whether the indicated order type is compatible with the implied order type and the fluid pump comprises, responsive to determining that the implied order type comprises the subsequent order: determining, responsive to determining that the indicated order type comprises the initial order or the subsequent order, that the indicated order type is compatible with the implied order type and the fluid pump; and determining, responsive to determining that the indicated order type comprises the modification order, the bolus order, or the secondary order, that the indicated order type is incompatible with the implied order type or the fluid pump.
Clause 7. The infusion device of any one of Clauses 3, 4, or 6, wherein determining whether the indicated order type is compatible with the implied order type and the fluid pump comprises, responsive to determining that the implied order type comprises the modification order: determining, responsive to determining that the indicated order type comprises the modification order, that the indicated order type is compatible with the implied order type and the fluid pump; and determining, responsive to determining that the indicated order type comprises the initial order, the subsequent order, the bolus order, or the secondary order, that the indicated order type is incompatible with the implied order type or the fluid pump.
Clause 8. The infusion device of any one of Clauses 3, 4, 6, or 7, wherein determining whether the indicated order type is compatible with the implied order type and the fluid pump comprises, responsive to determining that the implied order type comprises the bolus order: responsive to determining that the indicated order type comprises the bolus order: determining whether the fluid pump is configured to support the bolus order; determining, responsive to determining that the fluid pump is configured to support the bolus order, that the indicated order type is compatible with the implied order type and the fluid pump; and determining, responsive to determining that the fluid pump is not configured to support the bolus order, that the indicated order type is incompatible with the implied order type or the fluid pump; and determining, responsive to determining that the indicated order type comprises the initial order, the subsequent order, the modification order, or the secondary order, that the indicated order type is incompatible with the implied order type or the fluid pump.
Clause 9. The infusion device of any one of Clauses 3, 4, or 6 through 8, wherein determining whether the indicated order type is compatible with the implied order type and the fluid pump comprises, responsive to determining that the implied order type comprises the secondary order: responsive to determining that the indicated order type comprises the secondary order: determining whether the fluid pump is configured to support the secondary order; determining, responsive to determining that the fluid pump is configured to support the secondary order, that the indicated order type is compatible with the implied order type and the fluid pump; and determining, responsive to determining that the fluid pump is not configured to support the secondary order, that the indicated order type is incompatible with the implied order type or the fluid pump; and determining, responsive to determining that the indicated order type comprises the initial order, the subsequent order, the modification order, or the bolus order, that the indicated order type is incompatible with the implied order type.
Clause 10. The infusion device of any one of Clauses 1 through 9, wherein the indicated order type is incompatible with the implied order type or the fluid pump, and the processor is further configured to, responsive to determining that the indicated order type is incompatible with the implied order type or the fluid pump: determine (i) that the operating parameters of the APR are incompatible with the fluid pump, or (ii) that the implied order type is incompatible with an active infusion type of the fluid pump; and display, responsive to determining that the operating parameters of the APR are incompatible with the fluid pump, a first notice at a display device associated with the infusion device, the first notice indicating (i) that the APR is rejected and (ii) that the operating parameters of the APR are incompatible with the fluid pump; or display, responsive to determining that the implied order type is incompatible with the active infusion type of the fluid pump, a second notice at the display device, the second notice indicating (i) that the APR is rejected, (ii) that the implied order type is incompatible with the active infusion type of the fluid pump, and (iii) the active infusion type of the fluid pump.
Clause 11. A computer-implemented method for validating APRs, the method comprising: receiving an APR from a device manager, the APR comprising (i) an indicated order type comprising an initial order, a subsequent order, a modification order, a bolus order, or a secondary order, (ii) an identifier of a fluid pump of an infusion device, (iii) operating parameters including a requested fluid type and a requested dosage amount, and (iv) a command to automatically program the infusion device according to the operating parameters; detecting an activity state of the fluid pump, the activity state comprising (i) an idle state that indicates the fluid pump is not pumping a fluid or (ii) an active state that indicates the fluid pump is pumping a fluid; determining an implied order type for the APR based in part on the detected activity state, wherein the implied order type comprises an initial order, a subsequent order, a modification order, a bolus order, or a secondary order; determining whether the indicated order type is compatible with the implied order type and the fluid pump based in part on whether the indicated order type matches the implied order type; responsive to determining that the indicated order type is compatible with the implied order type and the fluid pump, (i) sending an acknowledgment message to the device manager and (ii) programming the fluid pump according to the operating parameters; and responsive to determining that the indicated order type is incompatible with the implied order type or the fluid pump, (i) sending a non-acknowledgment message and an error message to the device manager, and (ii) rejecting the command to automatically program the fluid pump according to the operating parameters.
Clause 12. The computer-implemented method of Clause 11, wherein the operating parameters further comprise requested dosing units, and determining the implied order type comprises: determining, responsive to detecting that the activity state of the fluid pump comprises the idle state, that the implied order type comprises the initial order; determining, responsive to detecting that the activity state comprises the active state, whether the requested fluid type matches an active fluid type being pumped by the fluid pump; determining, responsive to determining that the requested fluid type does not match the active fluid type, that the implied order type comprises the secondary order; determining, responsive to determining that the requested fluid type matches the active fluid type, whether the requested dosing units match active dosing units of the fluid pump; determining, responsive to determining that the requested dosing units do not match the active dosing units, that the implied order type comprises the bolus order; determining, responsive to determining that the requested dosing units match the active dosing units, whether the operating parameters of the APR also include a requested VTBI; determining, responsive to determining that the operating parameters do not include the requested VTBI, that the implied order type comprises the modification order; and determining, responsive to determining that the operating parameters include the requested VTBI, that the implied order type comprises the subsequent order.
Clause 13. The computer-implemented method of either of Clauses 11 or 12, further comprising: determining, responsive to detecting that the activity state comprises the active state, an active infusion type of the fluid pump based in part on active dosing units or an active fluid type, the active infusion type comprising a fluid infusion, a continuous infusion, or an intermittent infusion; wherein determining whether the indicated order type is compatible with the implied order type and the fluid pump is further based on the active infusion type.
Clause 14. The computer-implemented method of Clause 13, wherein determining the active infusion type comprises: determining whether active dosing units of the fluid pump comprise intermittent units; responsive to determining that the active dosing units comprise the intermittent units, determining that the active infusion type comprises the intermittent infusion; responsive to determining that the active dosing units do not comprise the intermittent units: determining whether the active dosing units consist of volume units; responsive to determining that the active dosing units do not consist of the volume units, determining that the active infusion type comprises the continuous infusion; and responsive to determining that the active dosing units consist of the volume units, determining whether the active fluid being pumped by the fluid pump is a drug, wherein the active infusion type comprises the fluid infusion when the active fluid is not a drug, and the active infusion type comprises the drug infusion when the active fluid is a drug.
Clause 15. The computer-implemented method of any one of Clauses 11 through 14, wherein determining whether the indicated order type is compatible with the implied order type and the fluid pump comprises, responsive to determining that the implied order type comprises an initial order: determining, responsive to determining that the indicated order type comprises the initial order or the subsequent order, that the indicated order type is compatible with the implied order type and the fluid pump; and determining, responsive to determining that the indicated order type comprises the modification order, the bolus order, or the secondary order, that the indicated order type is incompatible with the implied order type or the fluid pump.
Clause 16. The computer-implemented method of either of Clauses 13 or 14, wherein determining whether the indicated order type is compatible with the implied order type and the fluid pump comprises, responsive to determining that the implied order type comprises the subsequent order: determining, responsive to determining that the indicated order type comprises the initial order or the subsequent order, that the indicated order type is compatible with the implied order type and the fluid pump; and determining, responsive to determining that the indicated order type comprises the modification order, the bolus order, or the secondary order, that the indicated order type is incompatible with the implied order type or the fluid pump.
Clause 17. The computer-implemented method of any one of Clauses 13, 14, or 16, wherein determining whether the indicated order type is compatible with the implied order type and the fluid pump comprises, responsive to determining that the implied order type comprises the modification order: determining, responsive to determining that the indicated order type comprises the modification order, that the indicated order type is compatible with the implied order type and the fluid pump; and determining, responsive to determining that the indicated order type comprises the initial order, the subsequent order, the bolus order, or the secondary order, that the indicated order type is incompatible with the implied order type or the fluid pump.
Clause 18. The computer-implemented method of any one of Clauses 13, 14, 16, or 17, wherein determining whether the indicated order type is compatible with the implied order type and the fluid pump comprises, responsive to determining that the implied order type comprises the bolus order: responsive to determining that the indicated order type comprises the bolus order: determining whether the fluid pump is configured to support the bolus order; determining, responsive to determining that the fluid pump is configured to support the bolus order, that the indicated order type is compatible with the implied order type and the fluid pump; and determining, responsive to determining that the fluid pump is not configured to support the bolus order, that the indicated order type is incompatible with the implied order type or the fluid pump; and determining, responsive to determining that the indicated order type comprises the initial order, the subsequent order, the modification order, or the secondary order, that the indicated order type is incompatible with the implied order type or the fluid pump.
Clause 19. The computer-implemented method of any one of Clauses 13, 14, or 16through 18, wherein determining whether the indicated order type is compatible with the implied order type and the fluid pump comprises, responsive to determining that the implied order type comprises the secondary order: responsive to determining that the indicated order type comprises the secondary order: determining whether the fluid pump is configured to support the secondary order; determining, responsive to determining that the fluid pump is configured to support the secondary order, that the indicated order type is compatible with the implied order type and the fluid pump; and determining, responsive to determining that the fluid pump is not configured to support the secondary order, that the indicated order type is incompatible with the implied order type or the fluid pump; and determining, responsive to determining that the indicated order type comprises the initial order, the subsequent order, the modification order, or the bolus order, that the indicated order type is incompatible with the implied order type.
Clause 20. The computer-implemented method of any one of Clauses 11 through 19, further comprising, responsive to determining that the indicated order type is incompatible with the implied order type or the fluid pump: determining (i) that the operating parameters of the APR are incompatible with the fluid pump, or (ii) that the implied order type is incompatible with an active infusion type of the fluid pump; and displaying, responsive to determining that the operating parameters of the APR are incompatible with the fluid pump, a first notice at a display device associated with the infusion device, the first notice indicating (i) that the APR is rejected and (ii) that the operating parameters of the APR are incompatible with the fluid pump; or displaying, responsive to determining that the implied order type is incompatible with the active infusion type of the fluid pump, a second notice at the display device, the second notice indicating (i) that the APR is rejected, (ii) that the implied order type is incompatible with the active infusion type of the fluid pump, and (iii) the active infusion type of the fluid pump; wherein the indicated order type is incompatible with the implied order type or the fluid pump.
Clause 21. A non-transitory, computer-readable storage medium comprising instructions that, when executed by a processor of an electronic device, cause the electronic device to: receive an APR from a device manager, the APR comprising (i) an indicated order type comprising an initial order, a subsequent order, a modification order, a bolus order, or a secondary order, (ii) an identifier of a fluid pump of an infusion device, (iii) operating parameters including a requested fluid type and a requested dosage amount, and (iv) a command to automatically program the infusion device according to the operating parameters; detect an activity state of the fluid pump, the activity state comprising (i) an idle state that indicates the fluid pump is not pumping a fluid or (ii) an active state that indicates the fluid pump is pumping a fluid; determine an implied order type for the APR based in part on the detected activity state, wherein the implied order type comprises an initial order, a subsequent order, a modification order, a bolus order, or a secondary order; determine whether the indicated order type is compatible with the implied order type and the fluid pump based in part on whether the indicated order type matches the implied order type; responsive to determining that the indicated order type is compatible with the implied order type and the fluid pump, (i) send an acknowledgment message to the device manager and (ii) program the fluid pump according to the operating parameters; and responsive to determining that the indicated order type is incompatible with the implied order type or the fluid pump, (i) send an error message to the device manager, and (ii) reject the command to automatically program the fluid pump according to the operating parameters.
Clause 22. The non-transitory, computer-readable medium of Clause 21, further comprising instructions that, when executed by the processor, cause the electronic device to perform the computer-implemented method of any one of Clauses 12 through 20.

Further Consideration:
It is understood that the specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine-learning assessment model, or combinations thereof.

In any implementation, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An infusion device comprising:
a fluid pump; and
a processor configured to:
receive an automated programming request (APR) from a device manager, the APR comprising (i) an indicated order type, (ii) an identifier of the fluid pump, (iii) operating parameters including a requested fluid type and a requested dosage amount, and (iv) a command to automatically program the infusion device according to the operating parameters;
detect an activity state of the fluid pump, the activity state comprising (i) an idle state that indicates the fluid pump is not pumping a fluid or (ii) an active state that indicates the fluid pump is pumping a fluid;
determine an implied order type for the APR based in part on the detected activity state, wherein the implied order type comprises an initial order, a subsequent order, a modification order, a bolus order, or a secondary order;
determine whether the indicated order type is compatible with the implied order type and the fluid pump based in part on whether the indicated order type matches the implied order type;
responsive to determining that the indicated order type is compatible with the implied order type and the fluid pump, (i) send an acknowledgment message to the device manager and (ii) program the fluid pump according to the operating parameters; and
responsive to determining that the indicated order type is incompatible with the implied order type or the fluid pump, (i) send an error message to the device manager, and (ii) reject the command to automatically program the fluid pump according to the operating parameters.

2. The infusion device of claim 1, wherein the operating parameters further comprise requested dosing units, and determining the implied order type comprises:
responsive to detecting that the activity state of the fluid pump comprises the idle state, determining that the implied order type comprises the initial order;
responsive to detecting that the activity state comprises the active state, determining whether the requested fluid type matches an active fluid type being pumped by the fluid pump;
responsive to determining that the requested fluid type does not match the active fluid type, determining that the implied order type comprises the secondary order;
responsive to determining that the requested fluid type matches the active fluid type, determining whether the requested dosing units match active dosing units of the fluid pump;
responsive to determining that the requested dosing units do not match the active dosing units, determining that the implied order type comprises the bolus order;
responsive to determining that the requested dosing units match the active dosing units, determining whether the operating parameters of the APR also include a requested volume-to-be-infused (VTBI);
responsive to determining that the operating parameters do not include the requested VTBI, determining that the implied order type comprises the modification order; and
responsive to determining that the operating parameters include the requested VTBI, determining that the implied order type comprises the subsequent order.

3. The infusion device of claim 1, wherein:
the processor is further configured to, responsive to detecting that the activity state comprises the active state, determine an active infusion type of the fluid pump based in part on active dosing units or an active fluid type, the active infusion type comprising a fluid infusion, a continuous infusion, or an intermittent infusion; and
determining whether the indicated order type is compatible with the implied order type and the fluid pump is further based on the active infusion type.

4. The infusion device of claim 3, wherein determining the active infusion type comprises:
determining whether active dosing units of the fluid pump comprise intermittent units;
responsive to determining that the active dosing units comprise the intermittent units, determining that the active infusion type comprises the intermittent infusion;
responsive to determining that the active dosing units do not comprise the intermittent units:
determining whether the active dosing units consist of volume units;
responsive to determining that the active dosing units do not consist of the volume units, determining that the active infusion type comprises the continuous infusion; and
responsive to determining that the active dosing units consist of the volume units, determining whether the active fluid being pumped by the fluid pump is a drug, wherein the active infusion type comprises the fluid infusion when the active fluid is not a drug, and the active infusion type comprises the drug infusion when the active fluid is a drug.

5. The infusion device of claim 1, wherein determining whether the indicated order type is compatible with the implied order type and the fluid pump comprises, responsive to determining that the implied order type comprises an initial order:
responsive to determining that the indicated order type comprises the initial order or the subsequent order, determining that the indicated order type is compatible with the implied order type and the fluid pump; and
responsive to determining that the indicated order type comprises the modification order, the bolus order, or the secondary order, determining that the indicated order type is incompatible with the implied order type or the fluid pump.

6. The infusion device of claim 3, wherein determining whether the indicated order type is compatible with the implied order type and the fluid pump comprises, responsive to determining that the implied order type comprises the subsequent order:
responsive to determining that the indicated order type comprises the initial order or the subsequent order, determining that the indicated order type is compatible with the implied order type and the fluid pump; and
responsive to determining that the indicated order type comprises the modification order, the bolus order, or the secondary order, determining that the indicated order type is incompatible with the implied order type or the fluid pump.

7. The infusion device of claim 3, wherein determining whether the indicated order type is compatible with the implied order type and the fluid pump comprises, responsive to determining that the implied order type comprises the modification order:
responsive to determining that the indicated order type comprises the modification order, determining that the indicated order type is compatible with the implied order type and the fluid pump; and
responsive to determining that the indicated order type comprises the initial order, the subsequent order, the bolus order, or the secondary order, determining that the indicated order type is incompatible with the implied order type or the fluid pump.

8. The infusion device of claim 3, wherein determining whether the indicated order type is compatible with the implied order type and the fluid pump comprises, responsive to determining that the implied order type comprises the bolus order:
responsive to determining that the indicated order type comprises the bolus order:
determining whether the fluid pump is configured to support the bolus order;
responsive to determining that the fluid pump is configured to support the bolus order, determining that the indicated order type is compatible with the implied order type and the fluid pump; and
responsive to determining that the fluid pump is not configured to support the bolus order, determining that the indicated order type is incompatible with the implied order type or the fluid pump; and
responsive to determining that the indicated order type comprises the initial order, the subsequent order, the modification order, or the secondary order, determining that the indicated order type is incompatible with the implied order type or the fluid pump.

9. The infusion device of claim 3, wherein determining whether the indicated order type is compatible with the implied order type and the fluid pump comprises, responsive to determining that the implied order type comprises the secondary order:
responsive to determining that the indicated order type comprises the secondary order:
determining whether the fluid pump is configured to support the secondary order;
responsive to determining that the fluid pump is configured to support the secondary order, determining that the indicated order type is compatible with the implied order type and the fluid pump; and
responsive to determining that the fluid pump is not configured to support the secondary order, determining that the indicated order type is incompatible with the implied order type or the fluid pump; and
responsive to determining that the indicated order type comprises the initial order, the subsequent order, the modification order, or the bolus order, determining that the indicated order type is incompatible with the implied order type.

10. The infusion device of claim 1, wherein the indicated order type is incompatible with the implied order type or the fluid pump, and the processor is further configured to, responsive to determining that the indicated order type is incompatible with the implied order type or the fluid pump:
determine (i) that the operating parameters of the APR are incompatible with the fluid pump, or (ii) that the implied order type is incompatible with an active infusion type of the fluid pump; and
display, responsive to determining that the operating parameters of the APR are incompatible with the fluid pump, a first notice at a display device associated with the infusion device, the first notice indicating (i) that the APR is rejected and (ii) that the operating parameters of the APR are incompatible with the fluid pump; or
display, responsive to determining that the implied order type is incompatible with the active infusion type of the fluid pump, a second notice at the display device, the second notice indicating (i) that the APR is rejected, (ii) that the implied order type is incompatible with the active infusion type of the fluid pump, and (iii) the active infusion type of the fluid pump.

11. A computer-implemented method for validating APRs, the method comprising:
receiving an APR from a device manager, the APR comprising (i) an indicated order type, (ii) an identifier of a fluid pump of an infusion device, (iii) operating parameters including a requested fluid type and a requested dosage amount, and (iv) a command to automatically program the infusion device according to the operating parameters;
detecting an activity state of the fluid pump, the activity state comprising (i) an idle state that indicates the fluid pump is not pumping a fluid or (ii) an active state that indicates the fluid pump is pumping a fluid;
determining an implied order type for the APR based in part on the detected activity state, wherein the implied order type comprises an initial order, a subsequent order, a modification order, a bolus order, or a secondary order;
determining whether the indicated order type is compatible with the implied order type and the fluid pump based in part on whether the indicated order type matches the implied order type;
responsive to determining that the indicated order type is compatible with the implied order type and the fluid pump, (i) sending an acknowledgment message to the device manager and (ii) programming the fluid pump according to the operating parameters; and
responsive to determining that the indicated order type is incompatible with the implied order type or the fluid pump, (i) sending a non-acknowledgment message and an error message to the device manager, and (ii) rejecting the command to automatically program the fluid pump according to the operating parameters.

12. The computer-implemented method of claim 11, wherein the operating parameters further comprise requested dosing units, and determining the implied order type comprises:
responsive to detecting that the activity state of the fluid pump comprises the idle state, determining that the implied order type comprises the initial order;
responsive to detecting that the activity state comprises the active state, determining whether the requested fluid type matches an active fluid type being pumped by the fluid pump;
responsive to determining that the requested fluid type does not match the active fluid type, determining that the implied order type comprises the secondary order;
responsive to determining that the requested fluid type matches the active fluid type, determining whether the requested dosing units match active dosing units of the fluid pump;
responsive to determining that the requested dosing units do not match the active dosing units, determining that the implied order type comprises the bolus order;
responsive to determining that the requested dosing units match the active dosing units, determining whether the operating parameters of the APR also include a requested VTBI;
responsive to determining that the operating parameters do not include the requested VTBI, determining that the implied order type comprises the modification order; and
responsive to determining that the operating parameters include the requested VTBI, determining that the implied order type comprises the subsequent order.

13. The computer-implemented method of claim 11, further comprising:
responsive to detecting that the activity state comprises the active state, determining an active infusion type of the fluid pump based in part on active dosing units or an active fluid type, the active infusion type comprising a fluid infusion, a continuous infusion, or an intermittent infusion;
wherein determining whether the indicated order type is compatible with the implied order type and the fluid pump is further based on the active infusion type.

14. The computer-implemented method of claim 13, wherein determining the active infusion type comprises:
determining whether active dosing units of the fluid pump comprise intermittent units;
responsive to determining that the active dosing units comprise the intermittent units, determining that the active infusion type comprises the intermittent infusion;
responsive to determining that the active dosing units do not comprise the intermittent units:
determining whether the active dosing units consist of volume units;
responsive to determining that the active dosing units do not consist of the volume units, determining that the active infusion type comprises the continuous infusion; and
responsive to determining that the active dosing units consist of the volume units, determining whether the active fluid being pumped by the fluid pump is a drug, wherein the active infusion type comprises the fluid infusion when the active fluid is not a drug, and the active infusion type comprises the drug infusion when the active fluid is a drug.

15. The computer-implemented method of claim 11, wherein determining whether the indicated order type is compatible with the implied order type and the fluid pump comprises, responsive to determining that the implied order type comprises an initial order:
responsive to determining that the indicated order type comprises the initial order or the subsequent order, determining that the indicated order type is compatible with the implied order type and the fluid pump; and
responsive to determining that the indicated order type comprises the modification order, the bolus order, or the secondary order, determining that the indicated order type is incompatible with the implied order type or the fluid pump.

16. The computer-implemented method of claim 13, wherein determining whether the indicated order type is compatible with the implied order type and the fluid pump comprises, responsive to determining that the implied order type comprises the subsequent order:
responsive to determining that the indicated order type comprises the initial order or the subsequent order, determining that the indicated order type is compatible with the implied order type and the fluid pump; and
responsive to determining that the indicated order type comprises the modification order, the bolus order, or the secondary order, determining that the indicated order type is incompatible with the implied order type or the fluid pump.

17. The computer-implemented method of claim 13, wherein determining whether the indicated order type is compatible with the implied order type and the fluid pump comprises, responsive to determining that the implied order type comprises the modification order:
responsive to determining that the indicated order type comprises the modification order, determining that the indicated order type is compatible with the implied order type and the fluid pump; and
responsive to determining that the indicated order type comprises the initial order, the subsequent order, the bolus order, or the secondary order, determining that the indicated order type is incompatible with the implied order type or the fluid pump.

18. The computer-implemented method of claim 13, wherein determining whether the indicated order type is compatible with the implied order type and the fluid pump comprises, responsive to determining that the implied order type comprises the bolus order:
responsive to determining that the indicated order type comprises the bolus order:
determining whether the fluid pump is configured to support the bolus order;
responsive to determining that the fluid pump is configured to support the bolus order, determining that the indicated order type is compatible with the implied order type and the fluid pump; and
responsive to determining that the fluid pump is not configured to support the bolus order, determining that the indicated order type is incompatible with the implied order type or the fluid pump; and
responsive to determining that the indicated order type comprises the initial order, the subsequent order, the modification order, or the secondary order, determining that the indicated order type is incompatible with the implied order type or the fluid pump.

19. The computer-implemented method of claim 13, wherein determining whether the indicated order type is compatible with the implied order type and the fluid pump comprises, responsive to determining that the implied order type comprises the secondary order:
responsive to determining that the indicated order type comprises the secondary order:
determining whether the fluid pump is configured to support the secondary order;
responsive to determining that the fluid pump is configured to support the secondary order, determining that the indicated order type is compatible with the implied order type and the fluid pump; and
responsive to determining that the fluid pump is not configured to support the secondary order, determining that the indicated order type is incompatible with the implied order type or the fluid pump; and
responsive to determining that the indicated order type comprises the initial order, the subsequent order, the modification order, or the bolus order, determining that the indicated order type is incompatible with the implied order type.

20. A non-transitory, computer-readable storage medium comprising instructions that, when executed by a processor of an electronic device, cause the electronic device to:
receive an APR from a device manager, the APR comprising (i) an indicated order type, (ii) an identifier of a fluid pump of an infusion device, (iii) operating parameters including a requested fluid type and a requested dosage amount, and (iv) a command to automatically program the infusion device according to the operating parameters;
detect an activity state of the fluid pump, the activity state comprising (i) an idle state that indicates the fluid pump is not pumping a fluid or (ii) an active state that indicates the fluid pump is pumping a fluid;
determine an implied order type for the APR based in part on the detected activity state, wherein the implied order type comprises an initial order, a subsequent order, a modification order, a bolus order, or a secondary order;
determine whether the indicated order type is compatible with the implied order type and the fluid pump based in part on whether the indicated order type matches the implied order type;
responsive to determining that the indicated order type is compatible with the implied order type and the fluid pump, (i) send an acknowledgment message to the device manager and (ii) program the fluid pump according to the operating parameters; and
responsive to determining that the indicated order type is incompatible with the implied order type or the fluid pump, (i) send a non-acknowledgment message and an error message to the device manager, and (ii) reject the command to automatically program the fluid pump according to the operating parameters.
